(19) United States
(12) Patent Application Publication
(10) Pub. No.: US 2004/0254534 A1
(43) Pub. Date: Dec. 16, 2004

(54) Sliding connection assembly to facilitate lead stabilization

(76) Inventors: Bradford A. Bjorkman, Elk River, MN (US); Richard E. Stehr, Stillwater, MN (US)

Correspondence Address: HEIMBECHER & ASSOCIATES, LLC., 390 UNION BLVD, SUITE 650, LAKEWOOD, CO, 80228-6512, US

(21) Appl. No.: 10/460,783

(22) Filed: Jun. 11, 2003

(51) Int. Cl. A61M 5/178

(52) U.S. Cl. 604/160

(57) ABSTRACT

A connection system for connecting a hemostasis valve to a sheath is disclosed. The hemostasis valve has a cannula portion disposed on a distal end and an annular ledge protruding from an outer surface thereof. The sheath has a nipple extending proximally therefrom. The nipple also has an annular ledge disposed on an outer surface thereof and a nipple lumen adapted for receiving the distal end of the cannula portion. A sliding connector defining a connector lumen is disposed about the outer surface of the cannula portion and the outer surface of the nipple when the cannula portion is received in the lumen of the nipple. A connection between the hemostasis valve and the sheath is created by engaging the sliding connector with either or both of the annular ledge on the hemostasis valve and the annular ledge on the nipple.

### BACKGROUND OF INVENTION

a. Field of the Invention

This invention relates generally to the field of medical instruments used for intra-arterial and intravenous introduction and more specifically to a connection assembly for creating a fluid seal connection between such medical instruments.

b. Background Art

There are a number of medical procedures that require the introduction of medical instruments into arteries and veins. In one such procedure, known as the Seldinger procedure, a surgical opening is made in a vein or artery with a needle. A guide wire is then inserted through the lumen of the needle into the vein or artery. The needle is withdrawn, leaving the guide wire in place. A dilator is then inserted over the guide wire inside an associated sheath. The dilator and guidewire are removed once the sheath is in place. At this point, various types of catheters or leads may be inserted into the vessel within the lumen of the sheath using the sheath as a conduit to prevent damage to the vessel wall.

In certain medical procedures, for example, where a pacemaker lead is inserted into a patient, a sheath is normally used to guide the pacemaker lead to the appropriate location. Before the pacemaker lead is permanently secured in place and attached to a pacemaker, the sheath must be removed. Because of the size of its lumen, the sheath cannot simply slip over the exterior end of the pacemaker lead as that end of the lead contains a connector coupling for connection to the pacemaker.

Accordingly, there have been disclosed a number of splittable sheaths for use in the introduction of pacemaker lead. These sheaths can be split in half while still surrounding the pacemaker lead. In this use, once the pacemaker lead is in place, the sheath is longitudinally severed and removed from the pacemaker lead. For example, U.S. Pat. No. 4,983,168 discloses such a layered, peel-away hollow sheath, wherein the sheath wall is comprised of at least two layers, an inside cylindrical layer and an outside layer comprising two semi-cylindrical segments defining opposed axially-directed slits or slots therebetween, which comprise tear lines. U.S. Pat. No. 4,596,559 discloses a tear away sheath for use with a disposable introducer set in conjunction with a catheter. U.S. Pat. No. Re. 31,855 discloses a sheath that has an internal molecular orientation that tears easily in a lengthwise direction and with great difficulty in a crosswise or oblique direction. See also U.S. Pat. No. 4,581,025. Longitudinally scored or perforated sheaths are disclosed in U.S. Pat. Nos. 4,166,469; 4,243,050; 4,345,606; and 4,451,256.

Several problems may be encountered during the use of these splittable sheaths. For example, during the introduction of a pacemaker lead, a significant amount of bleeding may occur at the operation site, depending upon the blood pressure present in the vessel. Once the sheath is in place within a vessel, it provides a passageway for the free flow of blood away from the operation site. Further, because of this flow of blood, clotting may occur if the sheath remains in position for an extended period of time. These clots may cause emboli that may pass to the lung and have a detrimental impact on the patient. The use of sheaths may also provide a passageway for the introduction of air into the vessel. The inadvertent introduction of air into the blood system can cause air emboli in the patient that may have negative effects. Because of such problems, splittable sheaths are often removed from the theater of operation as soon as possible, even if it would be preferable to maintain them in position for a longer period of time. Such hurried procedures can result in errors or medical complications.

One method for restricting the flow of blood out of a sheath while a pacemaker lead is being introduced is for the physician to place his thumb over the exposed end of the sheath or to squeeze or pinch the exposed end of the sheath between his thumb and forefinger. However, neither of these methods for reducing the undesired flow of blood and air through the sheath is desirable, because the opportunity for loss of blood and introduction of air is still present. In addition, the structure of these sheaths still requires the surgeon to hold onto it while it is in place in the vessel, thereby limiting the surgeon's ability to perform other medical procedures at the same time. Moreover, squeezing the exposed end of the sheath can deform or even break the sheath, making lead insertion difficult and increasing the likelihood of damage to the lead as it passes through the sheath. Further, even when holding the end of the sheath or pinching the sheath, the flow of blood out of the sheath is not entirely stopped.

For these reasons, a hemostasis valve is often used in conjunction with a sheath to limit blood flow during the introduction of guide wires, catheters, pacemaker leads and other similar medical devices into the heart. This use of a hemostasis valve may, however, cause some issues. For example, because the exterior end of pacemaker leads is larger than the opening in conventional hemostasis valves, it is not possible for pacemaker leads to pass through these conventional hemostasis valves. In many cases the hemostasis valve is designed for use with a specific size of a catheter. Such hemostasis valves have been disclosed, for example, in U.S. Pat. Nos. 5,092,857 and 4,909,798. Another solution to this problem has been to provide splittable hemostasis valves integrally formed with splittable sheaths for the introduction of pacemaker leads as disclosed, for example, in U.S. Pat. Nos. 5,312,355 and 5,125,904. Similarly, splittable hemostasis valves that are not integral with a sheath, but merely connected thereto, may be used (see, for example, U.S. Pat. No. 6,083,207). A further solution to the problem has been to provide a “universal” hemostasis valve, wherein the valve assembly is designed to accommodate leads and catheters of a wide range of diameters.

A wide variety of circumstances can dictate which type of hemostasis valve is chosen for a particular application or in a particular situation. For example, the physician may want to delay introduction of a hemostasis valve onto a sheath until after the sheath is in position. This would suggest that an integral hemostasis valve and sheath is not desirable. In some circumstances, multiple leads or catheters of various diameters may need to be used. In these instances, particularly sized hemostasis valves would not be preferred. In other circumstances, the hemostasis valve may need to be removed during the operation, or perhaps removed and replaced several different times while the sheath remains in place. Such use might counsel against a splittable hemostasis valve that is no longer functional once split. Further, it is sometimes necessary to remove the hemostasis valve from the operating theater at a time when the sheath is still in use.

When the particular choice is made to use a non-splitting hemostasis valve, a further problem may arise that remains unaddressed by prior designs. Once introduced into the body intravascularly, leads are often placed in particular and sensitive positions and the intention is for the lead to remain in place. This is particularly true in the case of pacemaker leads that are imbedded in precise locations in the heart muscle to achieve particular results. The problem suggested occurs when attempting to remove a hemostasis valve from the lead. Sometimes the hemostasis valve is attached to the sheath with a Luer lock interface. When unscrewing the hemostasis valve, the friction fit between the valve assembly and the lead can cause the lead to rotate and either dislodge from or otherwise become misplaced about the heart muscle. Even when other fittings are used, the friction fit between the hemostasis valve and the heart muscle can cause the lead to become dislodged when removing the hemostasis valve.

The information included in this Background section of the specification, including any references cited herein and any description or discussion thereof, is included for technical reference purposes only and is not to be regarded as essential subject matter by which any claim of the present application or the scope of the invention is to be bound.

### SUMMARY OF INVENTION

The present invention is fundamentally a connection system for connecting a hemostasis valve to a sheath. The hemostasis valve has a cannula portion disposed on a distal end and an annular ledge protruding from an outer surface of the cannula portion. The annular ledge is disposed proximally from the distal end of the cannula portion. As used herein, “proximal” refers to the direction away from the patient and toward the physician, while “distal” indicates the direction toward the patient and away from the physician. The sheath has a nipple extending proximally therefrom. The nipple also has an annular ledge disposed on an outer surface thereof and a nipple lumen adapted for receiving the distal end of the cannula portion of the hemostasis valve. A sliding connector defining a connector lumen is disposed about the outer surface of the cannula portion of the hemostasis valve and the outer surface of the nipple of the sheath when the cannula portion is received in the lumen of the nipple. Further, either or both of the cannula portion and the nipple are received in the connector lumen. The sliding connector also slides proximally and distally along an interface between the cannula portion of the hemostasis valve and the nipple of the sheath when the cannula portion is received in the lumen of the nipple. To create a connection between the hemostasis valve and the sheath, the sliding connector engages either or both of the annular ledge on the hemostasis valve and the annular ledge on the nipple of the sheath.

In one embodiment, the sliding connector may also have an annular lip extending radially inward decreasing the diameter of a portion of the connector lumen for engagement with and retention by either of the annular ledge of the hemostasis valve or the annular ledge on the nipple of the sheath. Further, the sliding connector may be an internally threaded nut and the ledge on the nipple may be an external thread for engagement with the internally threaded nut to connect the hemostasis valve and the sheath. Alternately, the sliding connector may be an internally threaded nut and the ledge on the hemostasis valve may be an external thread for engagement with the internally threaded nut to connect the hemostasis valve and the sheath.

In an alternative embodiment, the sliding connector may have at least two longitudinally-oriented, biased tabs. Each of said tabs may have a tooth extending radially inward for engaging at least one of the either the annular ledge on the hemostasis valve or the annular ledge on the nipple to connect the hemostasis valve and the sheath.

In one embodiment, the sheath may be a splittable sheath. In this configuration, the sliding connector has a proximal range of motion such that the sliding connector can be moved to a position entirely about the cannula portion to expose the nipple and allow the splittable sheath to be split. In another embodiment with a splittable sheath, the sliding connector has a distal range of motion such that the sliding connector can be moved to a position entirely disengaged from the cannula portion. In this embodiment, the sliding connector may further be adapted to split in half axially in response to radial force imparted by the nipple on an inner surface of the sliding connector defined by the connector lumen as the splittable sheath is split.

The invention can alternatively be viewed as a hemostasis valve adapted to be releasably connected to a sheath. From this perspective, the hemostasis valve has a cannula portion disposed on a distal end. There is also an annular ledge protruding from an outer surface of the cannula portion disposed proximally from the distal end of the cannula portion. A sliding connector defining a connector lumen is disposed about the outer surface of the cannula portion of the hemostasis valve, wherein the sliding connector slides proximally and distally along the cannula portion. The sliding connector has a proximal end and a distal end, an annular lip extending radially inward at the proximal end for engagement with and retention by the annular ledge, and an engagement means at the distal end for engaging an opposing mating component of a sheath.

The invention can likewise alternatively be viewed as a sheath adapted to be releasably connected to a hemostasis valve. From this perspective, the sheath has a nipple extending distally therefrom. The nipple has an annular ledge disposed on an outer surface thereof and a nipple lumen adapted for receiving the distal end of a cannula portion of a hemostasis valve. A sliding connector defining a connector lumen is disposed about the outer surface of the nipple of the sheath, wherein the sliding connector slides proximally and distally along the nipple. The sliding connector has a proximal end and a distal end, an annular lip extending radially inward at the distal end for engagement with and retention by the annular ledge, and an engagement means at the proximal end for engaging an opposing mating component of a hemostasis valve.

The benefit of using the connection system of the present invention is that once the sliding connector disengages the connection between a hemostasis valve and a sheath without imparting movement to either the hemostasis valve or the sheath, thereby reducing the possibility of dislodging the lead. Further, once the sliding connector has been disengaged and moved away from the interface between the hemostasis valve and the sheath, either the hemostasis valve or the sheath can be moved slightly, proximally or distally, respectively, in a longitudinal direction along the lead to expose part of the lead between the hemostasis valve and the sheath to be grasped by the physician. In this manner, the lead can be held securely and be prevented from twisting or being pulled and dislodging while the hemostasis valve is removed. It is preferred that the sheath be moved downward slightly to expose the lead as the sheath is generally much greater in diameter than the lead and such movement is unlikely to disturb the lead. In a circumstance where a splittable sheath is used, once the sliding connector is disengaged and moved, the physician can split the sheath a small amount to expose the lead and then grasp the lead to prevent movement while removing the hemostasis valve.

Other features, utilities, and advantages of various embodiments of the invention will be apparent from the following more particular description of embodiments of the invention as illustrated in the accompanying drawings and defined in the appended claims.

### SUMMARY OF DRAWINGS

FIG. 1 is an isometric view of a sliding connection assembly according to one embodiment of the invention joining a hemostatis valve to a splittable sheath, wherein the sliding connector is in a fastened, distal position and is further connected to an extension tube and stop cock assembly.

FIG. 2 is a cross-section of the sliding connection assembly of FIG. 1 as indicated.

FIG. 3 is a cross-section of the connection assembly of the embodiment of FIG. 1 with the sliding connector in an unfastened, proximal position.

FIG. 4 is an isometric view of the sliding connection assembly of FIG. 2 with a lead placed through the assembly and further connected to an extension tube and stop cock assembly, wherein the sheath is compressed distally.

FIG. 5 is an isometric view of the sliding connection assembly of FIG. 2 with a lead placed through the assembly and further connected to an extension tube and stop cock assembly, wherein the hemostasis valve component is lifted proximally.

FIG. 6 is an isometric view of the sliding connection assembly of FIG. 2 with a lead placed through the assembly and further connected to an extension tube and stop cock assembly, wherein the sheath is partially separated.

FIG. 7 is a is an isometric view of a sliding connection assembly according to another embodiment of the invention joining a hemostatis valve to a splittable sheath, wherein the sliding connector is in a fastened, proximal position and is further connected to an extension tube and stop cock assembly.

FIG. 8 is a cross-section of the sliding connection assembly of FIG. 7 as indicated.

FIG. 9 is an isometric view of the sliding connection assembly of the embodiment of FIG. 7 with the sliding connector in an unfastened, distal position.

FIG. 10 is a cross-section of the sliding connection assembly of FIG. 9 as indicated.

FIG. 11 is a partial isometric view of another embodiment of a sliding connection assembly wherein the sliding connector is provided with locking tabs.

FIG. 12 is a partial cross-section of the sliding connection assembly of FIG. 11 with the sliding connector in a fastened position.

FIG. 13 depicts the sliding connection assembly of FIG. 12 with the tabs in a depressed position to release the sliding connector from the fastened position.

FIG. 14 is a partial cross-section of another embodiment of a sliding connection assembly similar to FIGS. 11-13 further having two annular ledges on the nipple to provide two locking positions for the tabs.

FIG. 15 is a partial isometric view of another embodiment of a sliding connection assembly wherein the sliding connector is a deformable locking cap in a fastened position.

FIG. 16 depicts the deformable locking cap of FIG. 15 in a deformed position to allow the sliding connector to be unfastened.

FIG. 17 is a cross-section of the sliding connection assembly of FIG. 16 as indicated.

FIG. 18 is a partial isometric view of another embodiment of a sliding connection assembly in an unfastened position, in which the sliding connector may be fastened to the nipple via a bayonet mechanism.

FIG. 19 is a cross-section of a sliding connection assembly of the type of FIG. 18 depicting the method of fastening the sliding connector to the nipple of the sheath.

FIG. 20 is a cross-section of a sliding connection assembly of the type of FIG. 18 depicting the sliding connector in a fastened postion.

### DETAILED DESCRIPTION OF PREFERRED EMBODIMENTS

The following disclosure of the invention describes a sliding connection system for connecting a hemostasis valve to a sheath. By providing a sliding connector about either the hemostasis valve or the sheath to connect one component to the other, the sliding connector can be moved from a position of interference with the interface between the hemostasis valve and the sheath to allow a physician access to an indwelling lead. In this manner, the indwelling lead may be held steady by the physician while removing the hemostasis valve or the sheath, for example, when using a splittable sheath.

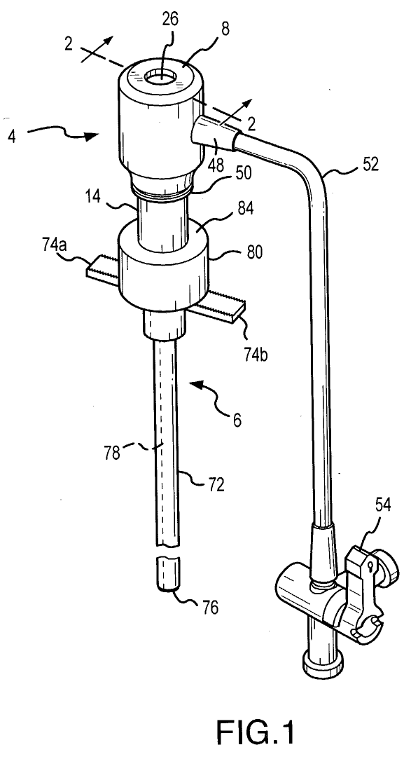

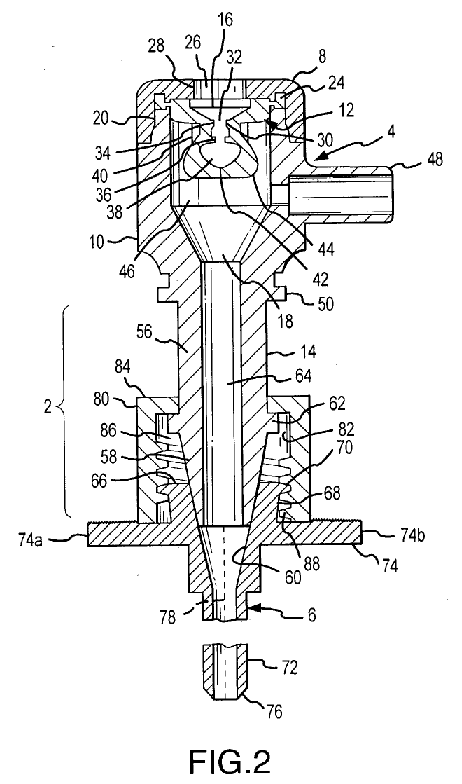

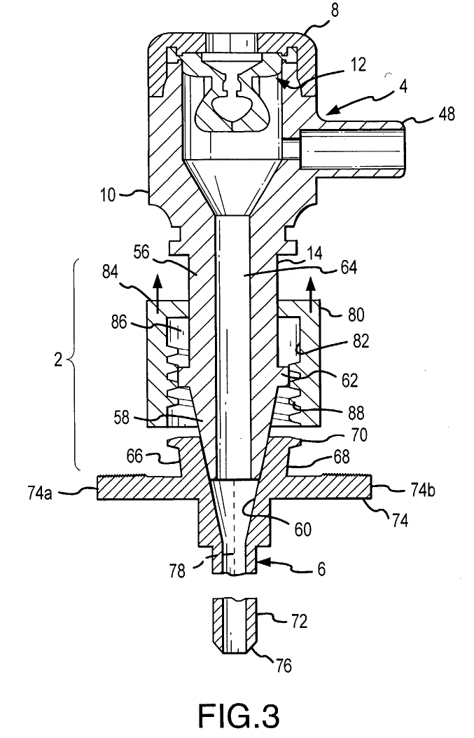

One embodiment of a sliding connection assembly 2 for use in conjunction with a separate hemostasis valve 4 and sheath 6 combination according to the present invention is depicted in FIG. 1 and in cross-section in two different positions in FIGS. 2 and 3. In FIG. 2 the sliding connection assembly 2 is shown fastened, connecting the hemostasis valve 4 to the sheath 6, and in FIG. 2 the connection assembly is shown released. The upper part of FIGS. 2 and 3 depict the hemostasis valve 4. The hemostasis valve 4 is formed from four major parts: a cap 8, a valve housing 10, a valve assembly 12, and a cannula portion 14. The cap 8 is attached to the top of the longitudinally extended valve housing 10. The valve housing has first and second opposing open ends 16, 18 to permit insertion of a catheter, dilator, guidewire, lead 90, or other instrument into and out of the interior of the valve housing 10. Hereinafter, the term “lead” will be used to refer generally to all instruments, including guidewires, leads, dilators, and catheters, that may be inserted into that hemostasis valve 4 and sheath 6.

The cap 8 and the valve housing 10 are formed from a relatively hard plastic, such as polycarbonate. The cap 8 may be secured to the valve housing 10, for example, by gluing, heat sealing, ultrasonic bonding, and by mechanically attaching to the valve housing, for example, threads, clips, or, as shown in the drawings, a snap fitting 20. The cap 8 and the valve housing 10 are first molded with respective interference fits and then may additionally be ultrasonically bonded together.

The hemostasis valve 4 also includes the valve assembly 12, which is formed from a pliant, resilient rubber such as silicone rubber or latex rubber having a durometer range of about 20-60 (Shore A), and which can be shaped to readily allow the passage of various sized leads 90. The valve assembly 12 may be of a one piece valve construction, although a two piece, moldable valve assembly may also be used. The hemostasis valve 4 also contains a cannula portion 14, which may be formed integrally with the hemostasis valve. The cannula portion 14 extends distally from the valve housing 10 and cooperates to provide an exit from the interior of the valve housing 10. The cannula portion 14 further includes engagement structures for interfacing with the sheath 6 and the sliding connection assembly 2.

As shown in FIGS. 2 and 3, the valve assembly 12, the cap 8, and the valve housing 10 are joined together by inserting the valve assembly 12 into the cap such that the uppermost edge 22 of the valve assembly 12 is fully inserted within the cap 8 and rests against a rib 24, which is preferably circular in nature. The cap 8 with the valve assembly 12 in position is then placed on top of the valve housing 10. The valve assembly 12 is inserted inside the valve housing 10, and downward pressure is applied to the cap 8 along with ultrasonic energy to bond the cap 8 to the valve housing 10. With the cap 8 and valve housing 10 engaged, downward pressure on the cap 8 is maintained causing compression of the uppermost edge of the valve assembly 12 by the rib 24, which serves to hold the valve assembly 2 in place within the valve housing 10.

The cap 8 is provided with a first opening 26 at the top, which can receive lead 90 that is inserted within the hemostasis valve 4 for purposes of introduction into body vessels. An exemplary valve assembly 12 has a conical receiving area 28 that tapers into a sealing neck 30 having a neck opening 32. Taken together the conical receiving area 28 and neck opening 32 provide for easy insertion of a lead 90 into the valve assembly 12 and through the neck opening 32. The sealing neck 30 may have a first narrowed portion 34 in communication with the conical receiving area 28, a second narrowed portion 36 in communication with a sealing chamber 38, and a broadened portion 40 between these first and second narrowed portions 34, 36.

The diameter of the opening of the first and second narrowed portions 34, 36 is slightly less than the diameter of a conventional lead 90 that will pass through this sealing neck 30. Preferably, the first narrowed portion 34 is slightly smaller than the second narrowed portion 36, although the first narrowed portion 34 may be larger than or the same diameter as the second narrowed portion 36. By reducing the amount of inner surface area of the sealing neck 30, which contacts a lead 90 as it passes through the passageway of the sealing neck 30, resistance to the movement of the lead 90 through the sealing neck 30 is also reduced. Notwithstanding this reduced resistance, a good seal is still created against bleeding because of the presence of the first and second narrowed portions 34, 36 of the sealing neck 30, which continue to press against the lead 90 as it passes through the hemostasis valve 4. The amount of the resistance to the movement of the lead 90 through the hemostasis valve 4 is directly related to the amount of material in the sealing neck 30 that contacts the lead 90 as it passes through the seal. By reducing the amount of this seal material to a minimum while at the same time retaining hemostasis around the lead 90 while passing through the sealing neck, good “feel” is provided while at the same time minimizing hemodynamic pressure dampening.

Communicating with the conical receiving area 28 and the neck opening 32 is the sealing chamber 38, which may be of any convenient shape, although preferably, it is semi-spherical or flattened spherical in shape. The interior diameter of the sealing chamber 38 is preferably the same as the largest outside diameter of any lead 90 that will be employed with the hemostasis valve 4. The diameter of the second narrowed portion 36 of the sealing neck 30, which is closest to the sealing chamber 38, may be slightly smaller than that of any lead 90 that will be employed so as to provide for sealing against the reverse flow of blood that may enter into the sealing chamber 38 while a lead 90 is in place in the hemostasis valve 4.

In order to provide support for the valve assembly 12 when a lead 90 is inserted through the sealing neck 30, support shoulders 40 may be located on the outside of the valve assembly 12 where the conical receiving area 28 tapers into the sealing neck 30 as shown in FIGS. 2 and 3. The support shoulders 40 do not extend outward beyond the widest portion of the sealing chamber 38 or downward around the outside surface of the sealing chamber 38 to increase the overall diameter of the valve assembly 12. Specifically, the support shoulders 40 do not increase the outside diameter of that portion of the valve assembly 12 containing the sealing chamber 30. Also, the support shoulders 40 do not extend downward beyond the widest portion of the outside of the sealing chamber 30, thus avoiding undue expansion of the neck opening 32 against the side walls of the valve housing 10 upon insertion of a large diameter lead 90. As a result, when a lead 90 is inserted through the neck opening 32, the sealing neck 30 will not unduly bulge out and come into contact with the walls of the valve housing 10. The support shoulders 40 also prevent the valve assembly 12 from extending excessively downward toward the second open end 18 of the valve housing 10 and, importantly, provide support for the seal on insertion and removal of leads 90 through the valve.

A single slit 42 in the valve assembly 12 creates opposing sealing lips 44 that are forced open by a lead 90 inserted into the body of the hemostasis valve 4. The spacial geometry of the walls of the semi-spherical sealing chamber 38 strongly force opposing sealing lips 44 into a normally closed position and hold them in that position to prevent an external reverse flow of blood. Likewise, when the sealing lips 44 are opened after a lead 90 is inserted, the opposing forces of the sealing neck 30 seal around the lead 90 and halt the reverse flow of blood.

The valve housing 10 is longitudinally extended to form a valve chamber 46. The first and second open ends 16, 18 of the valve housing 10 allow a lead 90 to be inserted through the valve chamber 46. Access to the interior of the valve chamber 46 may also be provided through a port 48 to which is attached tubing 52 and a fitting, for example, a stop cock 54, to permit insertion or withdrawal of fluids from the valve chamber 46 during use. The valve housing 10 of the hemostasis valve 4 may also be provided with a suture ring 50 to allow temporary attachment of the cannula portion 14 of the hemostasis valve 4 directly to a patient's body to provide stabilization of the hemostasis valve 4.

In one embodiment, shown in FIGS. 2-6, the cannula portion 14 of the hemostasis valve 4 may have a shaft 56 that is tapered at the distal end 58 for insertion into a symmetrically tapered lumen 60 in the proximal end 66 of a sheath 6. The cannula portion 14 may further have an annular ledge 62 extending circumferentially about the outer surface of the cannula portion 14. The annular ledge 62 may be spaced apart from the distal end 58 of the cannula portion 14, proximal to the point at which the cannula portion 14 begins to taper. The lumen 64 defined by the cannula portion 14 is constant throughout and is at least as large in diameter as the proximal first open end 16 of the hemostasis valve 4. The constant diameter of the cannula lumen 64 ensures adequate clearance for insertion and withdrawal of any leads 90.

A sheath 6 is provided as part of the connection system and is adapted at its proximal end 66 to interface with the hemostasis valve 4. In one embodiment, as shown in FIGS. 2-6, the sheath may have a nipple 68 on the proximal end 66. The nipple 68 is generally an annular wall defining a mating lumen 60. The nipple lumen 60 is tapered distally at the same grade of decreasing diameter as the outer surface of the tapered cannula portion 58. With these opposing tapered structures, the cannula portion 14 of the hemostasis valve 4 may be seated within the nipple lumen 60 to create a fluid-tight interface. The nipple 68 is further formed with an annular ledge 70 extending circumferentially about the outer surface of the nipple 68. The annular ledge 70 may be positioned at the proximal edge of the nipple 68 as shown or in any alternate position distal therefrom.

The sheath 6 may be a generally elongated, substantially cylindrical tube 72 having a handle 74 fixed to its proximal end 66. It may be formed by extrusion of any suitable plastic material, preferably a polyethylene or tetrafloroethylene plastic such as Pebax® (AUTOFINA Chemicals, Inc., Philadelphia, Pa.), wherein the plastic is compatible with body fluids, particularly blood. As shown in FIGS. 1-10, the sheath 6 may also be designed to split in half and tear apart from about an indwelling lead 90. The tube 72 has a proximal end 66 and a distal end 76 and mechanically formed, longitudinally extending zones of reduced thickness (shown in dash) defined by internally scored, longitudinally shallow grooves 78 or indentations running the length of the tube. The handle 74 preferably includes a pair of handle members 74a, 74b projecting perpendicularly outward from the cylindrically shaped tube 72. Upon pressure being placed against the top of the handle members 74a, 74b and outward radial force of pulling, the tube 72 splits for removal from the about a lead 90 previously inserted within.

In the embodiment of FIGS. 1-6, the sliding connection 2 assembly is provided by a sliding connector 80 disposed about the outer surface of the cannula portion 14 of the hemostasis valve 4. In general, the sliding connector 80 according the present invention comprises an annular wall 82 defining a connector lumen 86 disposed about the outer surface of the cannula portion 14. An annular lip 84 extends radially inward reducing the diameter of the connector lumen 86 at the proximal end of the sliding connector 80. The annular lip 84 is positioned on the proximal side of the annular ledge 62 of the cannula portion 14 for engagement with the proximal side of the annular ledge 62, thereby retaining the sliding connector 80 about the cannula portion 14, as shown in FIGS. 2 and 3. The sliding connector 80 slides proximally and distally along the cannula portion 14 within the bounds allowed distally by the retention of the annular ledge 62.

The sliding connector 80 further has an engagement structure at the distal end for engaging an opposing mating component of the nipple 68 on the sheath 6. In the embodiment of FIGS. 1-6, the sliding connector 80 may take the form of a Luer lock with internal threads 88 on the interior surface of the sliding connector 80 defined by the connector lumen 86. When the cannula portion 14 of the hemostasis valve 4 is seated within the nipple lumen 60, the sliding connector 80 may be moved distally along the cannula portion 14 to cover the interface between the cannula portion 14 and the nipple 68. The internal threads 88 of the sliding connector 80 may then be engaged with the annular ledge 70 of the nipple 68, which in this embodiment functions as an opposing male thread to the female threading 88 of the sliding connector 80. By rotating the sliding connector 80 about the cannula potion 14 and the nipple 68, the sliding connector 80 may be tightened against the annular ledge 70 of the nipple 68. The sliding connector 80 is fully engaged when the annular lip 84 of the sliding connector 80 is firmly seated against the annular ledge 62 of the cannula portion 14 and the hemostasis valve 4 is unable to move proximally or distally with respect to the sheath 6, as shown in FIGS. 1 and 2.

In one method of operation according to the embodiment of FIGS. 1-6, a needle is inserted into a patient's blood vessel. A guide wire is threaded through the lumen of the needle into the vessel. The needle is then removed leaving the guide wire in the vessel with a portion exposed. A dilator and splittable sheath 6 are then advanced together over the guide wire into the vessel. The dilator tip, which is tapered, increases the size of the opening in the blood vessel as it enters the vessel so that ultimately an opening large enough to accommodate the sheath 6 is formed. After the sheath 6 is inserted into the blood vessel, the dilator and guidewire are removed. The distal end of a medical device, such as a pacemaker lead 90, is then advanced through the splittable sheath 6, into the location within the patient for its utilization.

The hemostasis valve 4, depending upon the type (e.g., single or “universal” diameter) may be attached to the sheath 6 at any time during the medical procedure, before or after the lead 90 is placed in the sheath. With the hemostasis valve in place, it is possible to insert leads 90 having a wide range of diameters with ease. A lead 90 may be inserted through the first opening in the cap 8 and into the valve assembly 12. If the lead 90 is inserted slightly off center, it will be guided to the neck opening 32 by means of the conical receiving area 28. The lead 90 may then be moved through the passageway of the sealing neck 30 into the semi-spherical sealing chamber 38 and out through the sealing lips 44. After exiting through the sealing lips 44, the lead 90 is advanced through the cannula portion 14, out the opening, down through the sheath 6, and into the blood vessel. Any blood that flows between the sheath 6 and the lead 90 and up into the interior of the valve chamber 46 is not permitted to escape to the exterior because of the sealing action of the narrowed portion or portions of the sealing neck 30 around the body of the lead 90.

Alternatively, if a lead 90 is in place, the hemostasis valve 4 may be placed over the lead 90 and the tapered end 58 of the cannula portion 14 may be inserted into the lumen 60 of the nipple 68 extending proximally from the sheath 6. The hemostasis valve 4 may then be then secured onto the nipple 68 by sliding the connector 80 distally and rotating it such that the internal threads 88 of the sliding connector 80 engage the annular ledge 70 of the nipple 68. In this manner, the sliding connector 80 can be tightened against the nipple 68 until the internal annular lip 84 of the sliding connector 80 seats firmly against the annular ledge 62 of the cannula portion 14 (as shown in FIG. 2), thereby creating a fluid tight seal between the hemostasis valve 4 and the sheath 6.

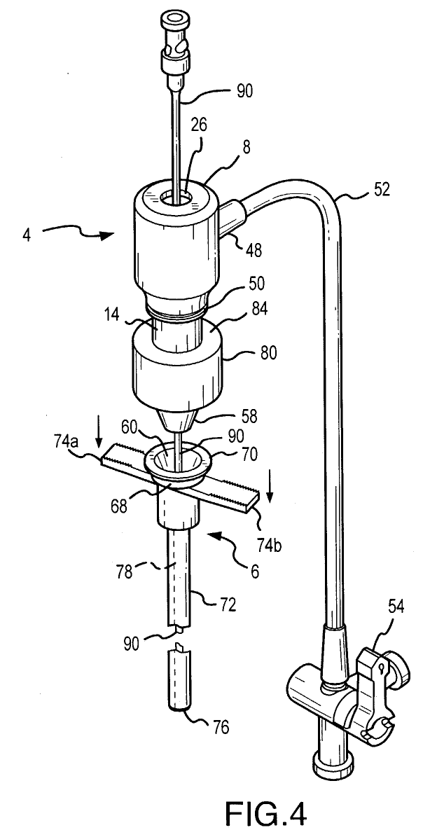

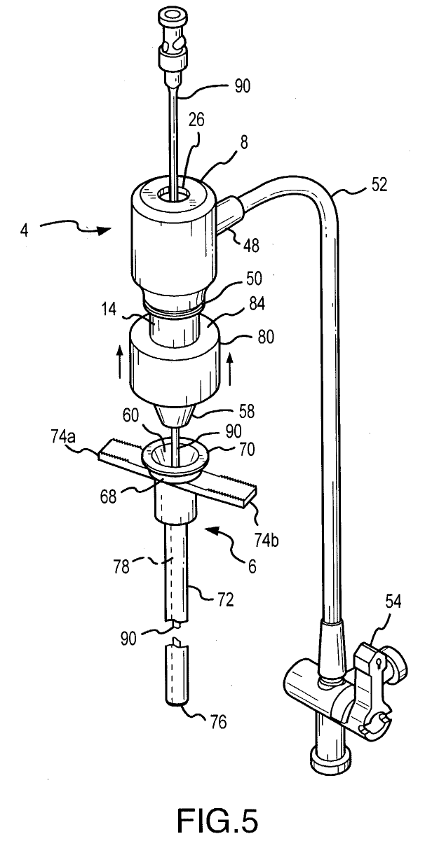

When it is time for the physician to remove the hemostasis valve 4, several options are possible. Each of these options first requires disengaging the sliding connector 80 from the nipple 68 by rotating the sliding connector 80 in the opposite direction than the direction used for engaging and securing the hemostasis valve 4 to the sheath 6. Once disengaged, the sliding connector 80 may then be move proximally, axially along the cannula portion 14 to reveal the interface between the cannula portion 14 and the sheath 6, as shown in FIGS. 3-6. The physician now has several options to remove the hemostasis valve 4 with minimal effect on the position of the lead 90. The first option, as shown in FIG. 4, is to push the sheath 6 distally a small amount to unseat the tapered end 58 of the cannula portion 14 from the receptacle in the nipple 68 and expose a section of the lead 90. As the sheath 6 is generally made from a pliable material, minor compression of the sheath 6 is easily achieved without moving the lead 90. The physician can then grasp the exposed section of the lead 90 between his fingers or with an instrument and hold the lead 90 steady while removing the hemostasis valve 4. Alternately, although less desirable because of the friction fit between the valve assembly 12 and the lead 90, the hemostasis valve 4 may be moved slightly proximally to similarly unseat the tapered end 58 of the cannula portion 14 from the receptacle in the nipple 68 and expose a section of the lead 90, as shown in FIG. 5. Such a small movement may have negligible impact upon the position of the lead 90. Again, the physician can then grasp the exposed section of the lead 90 between his fingers or with an instrument and hold the lead 90 steady while removing the hemostasis valve 4.

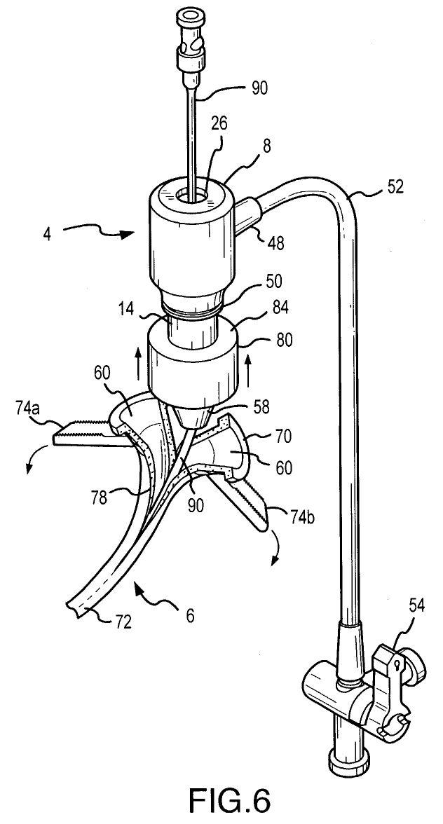

Another option is available in the circumstance that a splittable sheath 6 is used as shown in FIG. 6. Again, the sliding connector 80 is first disengaged from the nipple 68 and moved proximally, axially along the cannula portion 14 to reveal the interface between the cannula portion 14 and the sheath 6. The physician may then grasp the handle members 74a, 74b of the splittable sheath 6 and by applying force distally and outward radially, the sheath 6 will begin to split at the proximal end. Once the sheath 6 has split enough to expose the indwelling lead 90, the physician can then grasp the exposed section of the lead 90 between his fingers or with an instrument and hold the lead 90 steady while removing the hemostasis valve 4.

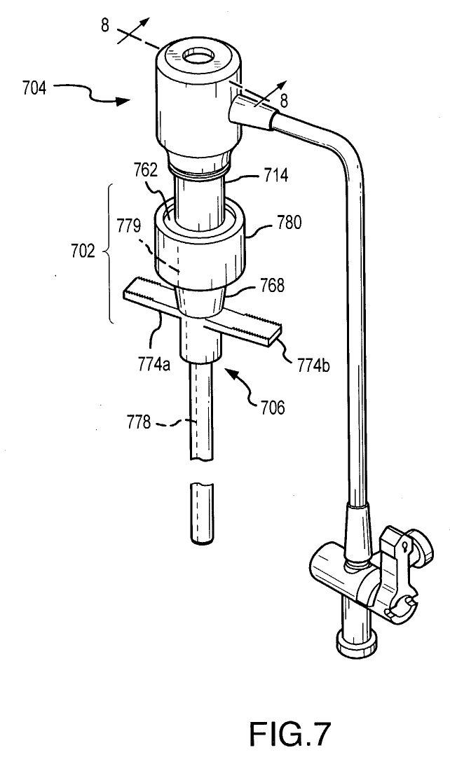

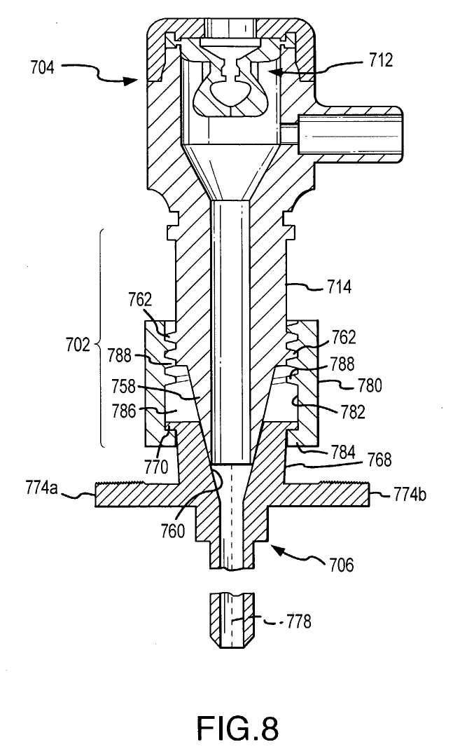

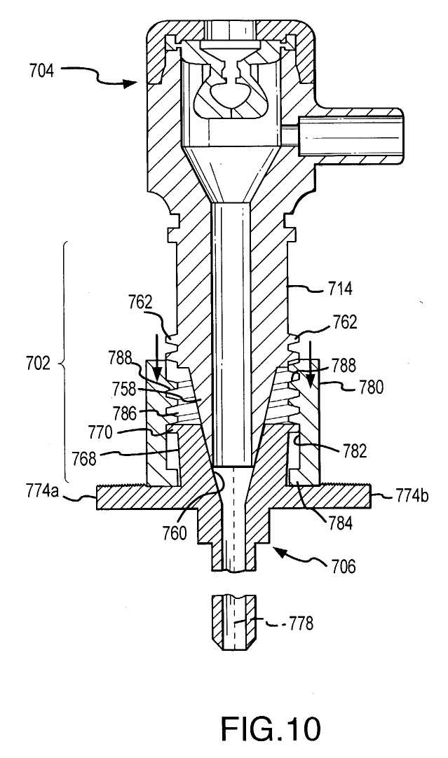

An alternative embodiment of the sliding connection assembly 702 is depicted in FIGS. 7-10. (Reference numerals associated with the embodiments of FIGS. 7-10 are similar to the reference numerals for FIGS. 1-6, but are preceded by the prefix “7” as an indication that there are some structural differences between the embodiments.) In this embodiment, the sliding connector 780 is retained about the outer surface of the nipple 768 of the sheath 706 rather than about the hemostasis valve 704. The sliding connector 780 is similarly provided in the form of an annular wall 782 defining a lumen 786 disposed about the outer surface of the nipple 768. An annular lip 784 extends radially inward reducing the diameter of the connector lumen 786 at the distal end of the sliding connector 780. The annular lip 784 is positioned on the distal side of the annular ledge 770 of the nipple 768 for engagement with the annular ledge 770, thereby retaining the sliding connector 780 about the nipple 768, as shown in FIGS. 8 and 10. The sliding connector 780 slides proximally and distally along the nipple 768 within the bounds allowed proximally by the retention of the annular ledge 770.

The sliding connector 780 further has an engagement structure at the proximal end for engaging an opposing mating component of the nipple 768 on the sheath 706. In the embodiment of FIGS. 7-10, the sliding connector 780 again may take the form of a Luer lock with internal threads 788 on the interior surface of the sliding connector 780 defined by the connector lumen 786. When the cannula portion 714 of the hemostasis valve 704 is seated within the nipple lumen 760, the sliding connector 780 may be moved proximally along the nipple 768 to engage the annular ledge 762 of the cannula portion 714, which in this embodiment functions as an opposing male thread to the female threading 788 of the sliding connector 780. By rotating the sliding connector 780 about the cannula potion 714 and the nipple 768, the sliding connector 780 may be tightened against the annular ledge 762 of the cannula portion 714. The sliding connector 780 is fully engaged when the annular lip 784 of the sliding connector 780 is firmly seated against the annular ledge 770 of the nipple 768, and the sheath 706 is unable to move proximally or distally with respect to the hemostasis valve 704, as shown in FIGS. 7 and 8.

With the connection assembly 702 of FIGS. 7-10, an alternative method is available for attaching and removing the hemostasis valve 704 from the sheath 706 and the indwelling lead 780. In this embodiment, the sliding connector 780 is moveably attached to the nipple 768 rather than the cannula portion 714 of the hemostasis valve 704. Once the tapered end 758 of the cannula portion 714 is received in the lumen 760 of the nipple 768, the sliding connector 780 is secured onto the cannula portion 714 by sliding the sliding connector 780 proximally and rotating it such that the internal threads 788 of the sliding connector 780 engage the annular ledge 762 of the cannula portion 714. In this manner, the sliding connector 780 can be tightened against the cannula portion 714 until the internal annular lip 784 of the sliding connector 780 seats firmly against the annular ledge 770 of the nipple 768 (as shown in FIG. 8), thereby creating a fluid tight seal between the hemostasis valve 704 and the sheath 706.

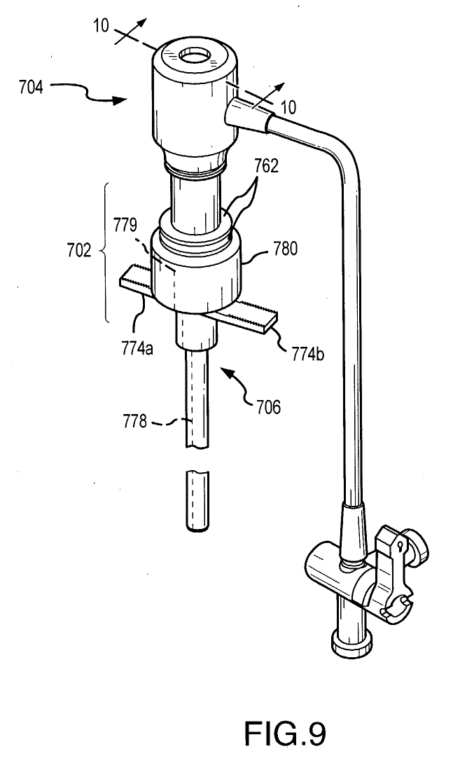

When time for the physician to remove the hemostasis valve 704, several options are again possible. Each of these options first requires disengaging the sliding connector 780 from the cannula portion 714 by rotating the sliding connector 780 in the opposite direction than the direction used for engaging and securing the hemostasis valve 704 to the sheath 706. Once disengaged, the sliding connector 780 may then be move distally, axially along the nipple 768, as shown in FIGS. 9 and 10. The physician now has the same options as before to remove the hemostasis valve 704 with minimal effect on the position of the lead 790. The first option is again to push the sheath 706 distally a small amount to unseat the tapered end 758 of the cannula portion 714 from the receptacle in the nipple 768 and expose a section of the lead 790. Alternately, although again less desirable because of the friction fit between the valve assembly 712 and the lead 790, the hemostasis valve 704 may be moved slightly proximally to similarly unseat the tapered end 758 of the cannula portion 714 from the receptacle in the nipple 768 and expose a section of the lead 790. In either case, the physician can then grasp the lead 790 between his fingers or with an instrument and hold the lead 790 steady while removing the hemostasis valve 704 proximally from about the lead 790.

In the circumstance that a splittable sheath 706 is used, as the sliding connector 780 in this embodiment is retained on the nipple 768 by the engagement between the annular lip 784 of the sliding connector 780 and the annular ledge 770 of the nipple 768, the sliding connector 780 likewise needs to be splittable. The sliding connector 780 is first disengaged from the annular ledge 762 of the cannula portion 714 and moved distally, axially along the nipple 768 until the sliding connector 780 seats against the handle members 774a, 774b similar to what is shown in FIG. 10. The scored longitudes 779 of the sliding connector 780 should be aligned with the scores 778 of the splittable sheath 706. The physician may then grasp the handle members 774a, 774b of the splittable sheath 706 and by applying force distally and outward radially, the sheath 706 will begin to split at the proximal end. The distal and radial force of the nipple 768 splitting and pressing on the interior walls defined by the lumen 786 of the sliding connector 780 cause the sliding connector 780 to likewise split. Once the sliding connector 780 falls away and the sheath 706 has split enough to expose the indwelling lead 790, the physician can then grasp the exposed section of the lead 790 between his fingers or with an instrument and hold the lead 790 steady while removing the hemostasis valve 704.

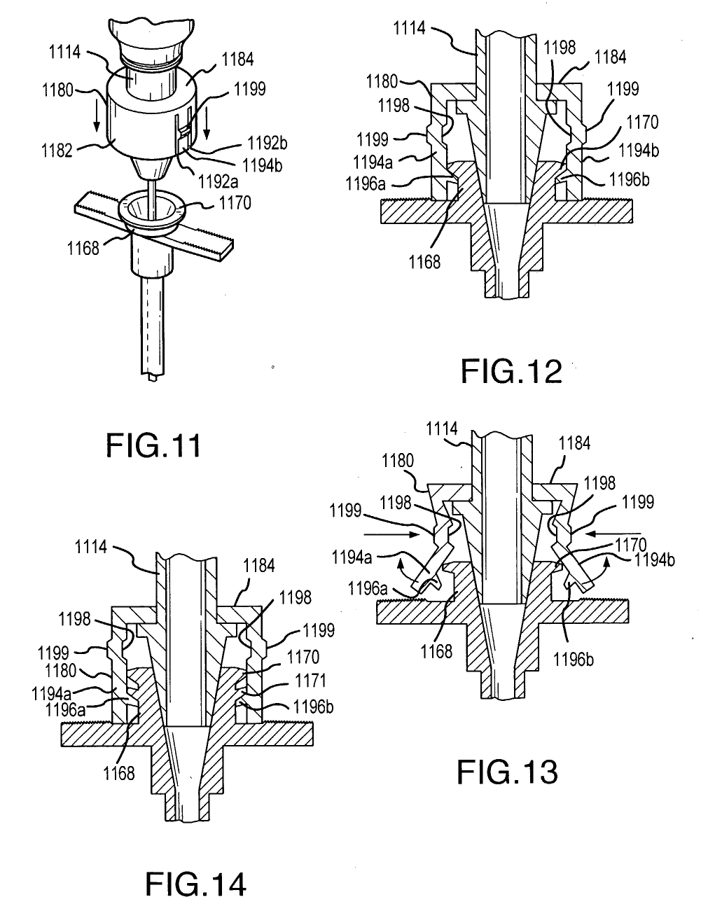

The connection assembly 2 and particularly the interface between the sliding connector 80 and the nipple 68 on the sheath 6 can take many different forms. For example, in another embodiment as shown in FIGS. 11-14, a sliding connector 1180 may similarly be an annular wall 1182 disposed about the cannula portion 1114. (Reference numerals associated with the embodiments of FIGS. 11-14 are similar to the reference numerals for FIGS. 1-6, but are preceded by the prefix “11” as an indication that there are some structural differences between the embodiments.) In this embodiment, parallel axial cut-outs 1192a, 1192b are provided in at least two opposing positions along the annular wall 1182 of the sliding connector 1180 from the distal end to a point spaced apart from the proximal end defining the annular lip 1184. In this manner, two opposing longitudinal, locking tabs 1194a, 1194b are formed. Each of the tabs 1194a, 1194b may have a tooth 1196a, 1196b on the distal end of the interior surface of the tab 1194a, 1194b defined by the connector lumen. Each tooth 1196a, 1196b is radially directed and is adapted to engage and be retained by the annular ledge 1170 of the nipple 1168. Each tab may have a plurality of teeth (not shown) extending proximally along the interior surface to provide for successive engagement by each tooth under axial pressure in the distal direction to provide a tighter seal between the cannula portion 1114 and the nipple 1168. Alternatively, the nipple 1168 may have a second annular ledge 1171, as shown in FIG. 14, to similarly provide for successive engagement by the teeth 1196a, 1196b on the tabs 1194a, 1194b of the sliding connector 1180.

The tabs 1194a, 1194b may be biased to allow for a firm connection with the annular ledge 1170 of the nipple 1168 and likewise for ease of release to remove the sliding connector 1180 from engagement with the annular ledge 1170 of the nipple 1168. An indentation 1198 may be formed on the interior surface of each tab and positioned proximal to the distally positioned teeth 1196a, 1196b. The indentations 1198 may act to create a natural hinge on each tab 1194a, 1194b when radial pressure is applied on the outer surface of each tab 1194a, 1194b, for example, by a physician squeezing each tab 1194a, 1194b between his fingers. In this manner the distal end of each tab 1194a, 1194b can be forced radially outward to disengage the teeth 1196a, 1196b from the annular ledge 1170 of the nipple 1168 and allow the sliding connector 1180 to be easily removed from the interface between the cannula portion 1114 and the nipple 1168. The annular ledge 1170 may act as a fulcrum to direct the distal ends of the tabs 1194a, 1194b radially outward as inward radial pressure is applied proximal to the indentations 1198. To aid in forcing the distal ends of the tabs 1194a, 1194b radially outward, a raised grip 1199 may be provided on the outer surface of each tab 1194a, 1194b proximal to the indentation 1198. The sliding connector 1180 of this embodiment may be limited to a single use or may be used to repeatedly fasten or unfasten the cannula portion 1114 to the nipple 1168 depending upon the resiliency of the locking tabs 1194a, 1194b. Resiliency may be affected by, for example, the depth of the indentation 1198 (i.e., the thickness of the tabs 1194a, 1194b at the location of the indentation) and the material properties of the plastic used to make the sliding connector 1180.

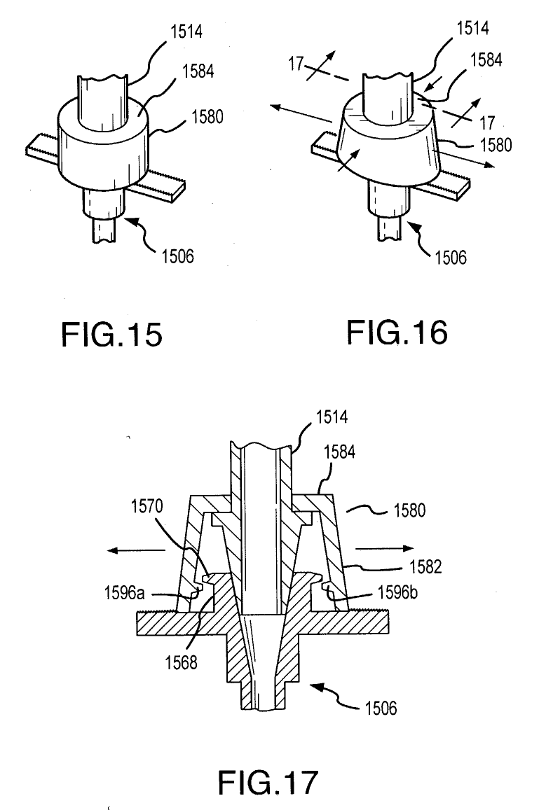

A further exemplary embodiment of a connection interface between the sliding connector 1580 and the nipple 1568 on the sheath 1506 is shown in FIGS. 15-17. (Reference numerals associated with the embodiments of FIGS. 11-14 are similar to the reference numerals for FIGS. 1-6, but are preceded by the prefix “15” as an indication that there are some structural differences between the embodiments.) In FIG. 15, the sliding connector 1580 is provided as a deformable cap. FIG. 16 depicts the sliding connector 1580 in a deformed position that allows the sliding connector 1580 to be removed from the nipple 1506. As shown in FIG. 17, the sliding connector 1580 has two locking teeth 1596a, 1596b disposed in opposing positions along the interior of annular wall 1582 of the sliding connector 1580. Each tooth 1596a, 1596b is radially directed and is adapted to engage and be retained by the annular ledge 1570 of the nipple 1568. To unfasten the sliding connector 1580 from the nipple 1568, the annular wall 1582 of the sliding connector 1580 is depressed radially inward at opposing positions intermediate to the locations of the teeth 1596a, 1596b as indicated by the arrows pointing radially inward in FIG. 16. While the annular lip 84 maintains its shape so as not to bind against the cannula portion 1514, the annular wall 1582 is deformed and the portions of the annular wall 1582 upon which the teeth 1596a, 1596b are disposed are forced radially outward, as shown by the arrows in FIG. 16. In this manner the teeth 1596a, 1596b are disengaged from the annular ledge 1570 of the nipple 1568 and the sliding connector 1580 may be disengaged from the nipple 1568 and slid upwardly about the cannula portion 1514.

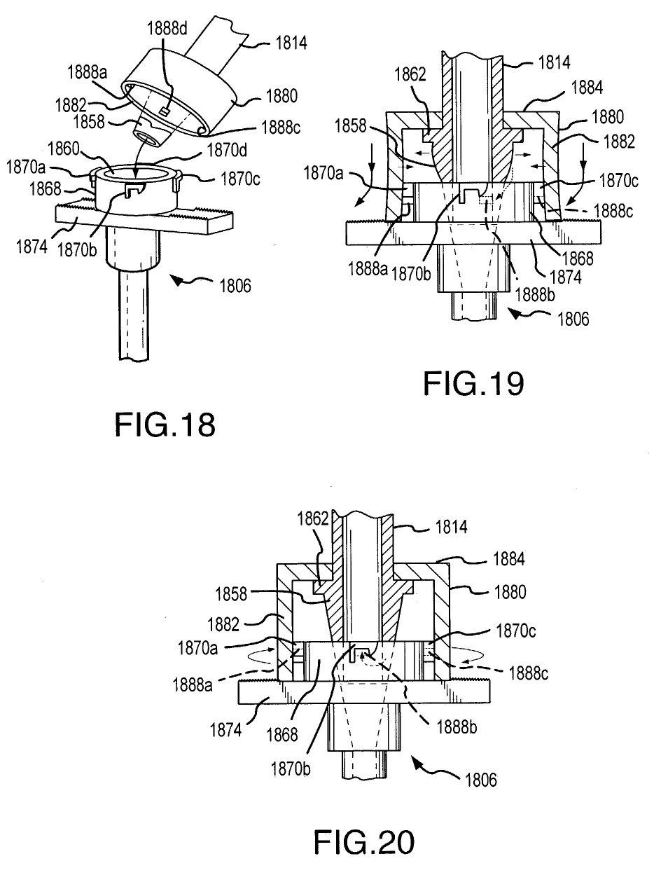

Another exemplary embodiment of a connection interface between the sliding connector 1880 and the nipple 1868 on the sheath 1806 is shown in FIGS. 18-20. (Reference numerals associated with the embodiments of FIGS. 18-20 are similar to the reference numerals for FIGS. 1-6, but are preceded by the prefix “18” as an indication that there are some structural differences between the embodiments.) As shown in FIGS. 18-20, the sliding connector 1580 has four locking teeth 1888a-d disposed at 90° separations along the interior of annular wall 1882 of the sliding connector 1880. Each tooth 1888a-d is radially directed and is adapted to engage and be retained by respective bayonet fittings 1870a-d similarly disposed about the outer surface of the nipple 1568 at 90° separations. The bayonet fittings 1870a-d are structural substitutes for and provided in lieu of the ledge 70 of FIGS. 1-6 to retain the sliding connector 1880. It should be recognized that as few as two teeth and corresponding bayonet fittings may be used to engage the sliding connector 1880 with the nipple 1868.

In order to engage the sliding connector 1880 with the nipple 1868, the sliding connector 1880 is rotated clockwise around the cannula portion 1814 until the teeth 1888a-d interface with the bayonet fittings 1870a-d. When engaging the teeth 1888a-d in respective bayonet fittings 1870a-d, the hooks of the bayonet fittings 1870 may slightly deform (not shown) to allow the teeth 1888a-d to move past the hooks and securely seat in the bayonet fittings 1870a-d as shown in FIG. 20. Alternately, the tapered end 1858 of the cannula portion 1814 and the annular walls 1882 of the sliding connector 1880 may slightly deform (as shown in FIG. 19) due to downward force on the teeth 1888a-d imparted at the interface between the bayonet fittings 1870a-d and the teeth 1888a-d, and translated by the interface between the annular lip 1884 of the sliding connector 1880 and the annular ledge 1862 of the cannula portion 1814, as the sliding connector 1880 is rotated. This likewise allows the teeth 1888a-d to move past the hooks and securely seat in the bayonet fittings 1870a-d as shown in FIG. 20 and further allows the annular walls 1882 of the sliding connector 1880 to seat against the handle member 1874 of the sheath 1806. To release the sliding connector 1880 from the nipple 1868, a slight amount of distal pressure may be applied to the sliding connector 1880 while rotating the sliding connector 1880 counterclockwise to slightly deform the tapered end 1862 of the cannula portion 1814 and the annular walls 1882 of the sliding connector 1880 and the allow the teeth 1888a-d to slip out of the seats in the bayonet fittings 1870a-d.

It should be apparent that the alternate embodiments of the sliding connector 80 and the connection interface with the nipple 68 described herein with respect to FIGS. 11-20 could likewise be used in the embodiment described herein with respect to FIGS. 7-10.

Although various embodiments of this invention have been described above with a certain degree of particularity, or with reference to one or more individual embodiments, those skilled in the art could make numerous alterations to the disclosed embodiments without departing from the spirit or scope of this invention. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A connection system for connecting a hemostatis valve to a sheath, the system comprising:
a hemostasis valve having a cannula portion disposed on a distal end and a first ledge protruding from an outer surface of the cannula portion and disposed proximally from the distal end of the cannula portion;
a sheath having a nipple extending proximally therefrom, wherein the nipple further comprises:
a second ledge disposed on an outer surface of the nipple;
a nipple lumen adapted for receiving the distal end of the cannula portion of the hemostasis valve; and
a sliding connector in the form of an annular wall defining a connector lumen and disposed about the outer surface of the cannula portion of the hemostasis valve and the outer surface of the nipple of the sheath when the cannula portion is received in the lumen of the nipple; wherein
at least one of the cannula portion and the nipple are received in the connector lumen;
the sliding connector slides proximally and distally along an interface between the cannula portion of the hemostasis valve and the nipple of the sheath when the cannula portion is received in the lumen of the nipple; and
the sliding connector further comprises an engagement means for engaging at least one of the first ledge and the second ledge to connect the hemostasis valve and the sheath.

2. The system of claim 1, wherein the sliding connector further comprises an annular lip extending radially inward from the annular wall decreasing the diameter of a portion of the connector lumen for engagement with and retention by at least one of the first ledge and the second ledge to connect the hemostasis valve and the sheath.

3. The system of claim 1, wherein the engagement means comprises an internal threading disposed on the annular wall and the second ledge comprises an external threading for engaging with the internal threading to connect the hemostasis valve and the sheath.

4. The system of claim 1, wherein the engagement means comprises an internal threading disposed on the annular wall and the first ledge comprises an external threading for engaging with the internal threading to connect the hemostasis valve and the sheath.

5. The system of claim 1, wherein the engagement means comprises at least two longitudinally-oriented tabs formed in the annular wall, each of said tabs having a tooth extending radially inward for engaging at least one of the first ledge and the second ledge to connect the hemostasis valve and the sheath.

6. The system of claim 1, wherein the engagement means comprises at least two teeth disposed upon the annular wall and extending radially inward for engaging at least one of the first ledge and the second ledge to connect the hemostasis valve and the sheath; and
wherein the annular wall is deformable to allow the at least two teeth to slide past the at least one of the first ledge and the second ledge when connecting the hemostasis valve and the sheath.

7. The system of claim 1, wherein the engagement means comprises at least two teeth disposed upon the annular wall and extending radially inward;
at least one of the first ledge and the second ledge comprises at least two bayonet fittings disposed to engage the at least two teeth, respectively.

8. The system of claim 1, wherein
the sheath is a splittable sheath; and
the sliding connector has a proximal range of motion such that the sliding connector can be moved to a position entirely about the cannula portion to expose the nipple and allow the splittable sheath to be split.

9. The system of claim 1, wherein
the sheath is a splittable sheath;
the sliding connector has a distal range of motion such that the sliding connector can be moved to a position entirely disengaged from the cannula portion; and
the sliding connector is adapted to be split in half axially in response to radial force imparted by the nipple on an inner surface of the sliding connector defined by the connector lumen as the splittable sheath is split.

10. A hemostasis valve adapted to be releasably connected to a sheath, the hemostatsis valve comprising:
a cannula portion disposed on a distal end;
a ledge protruding from an outer surface of the cannula portion and disposed proximally from the distal end of the cannula portion;
a sliding connector in the form of an annular wall defining a connector lumen and disposed about the outer surface of the cannula portion of the hemostasis valve, wherein the sliding connector slides proximally and distally along the cannula portion; and wherein the sliding connector further comprises:
a proximal end and a distal end;
an annular lip extending radially inward from the annular wall at the proximal end for engagement with and retention by the ledge; and
an engagement means at the distal end for engaging an opposing mating component of a sheath.

11. The hemostasis valve of claim 10, wherein the engagement means comprises an internal threading disposed on the annular wall.

12. The hemostasis valve of claim 10, wherein the engagement means comprises at least two longitudinally-oriented tabs formed in the annular wall, each of tabs having a tooth extending radially inward for engaging the opposing mating component of the sheath.

13. The system of claim 10, wherein the engagement means comprises at least two teeth disposed upon the annular wall and extending radially inward for engaging the opposing mating component of the sheath.

14. The system of claim 13, wherein the annular wall is deformable to allow the at least two teeth to slide past the opposing mating component of the sheath.

15. A sheath adapted to be releasably connected to a hemostasis valve, the sheath comprising:
a sheath having a nipple extending proximally therefrom, wherein the nipple further comprises:
a ledge disposed on an outer surface of the nipple;
a nipple lumen adapted for receiving a distal end of a cannula portion of a hemostasis valve;
a sliding connector in the form of an annular wall defining a connector lumen and disposed about the outer surface of the nipple, wherein the sliding connector slides proximally and distally along the nipple; and wherein the sliding connector further comprises:
a proximal end and a distal end;
an annular lip extending radially inward from the annular wall at the distal end for engagement with and retention by the ledge; and
an engagement means at the proximal end for engaging an opposing mating component of a hemostasis valve.

16. The sheath of claim 15, wherein the engagement means comprises an internal threading disposed on the annular wall.

17. The sheath of claim 15, wherein the engagement means comprises at least two longitudinally-oriented tabs formed in the annular wall, each of the tabs having a tooth extending radially inward for engaging the opposing mating component of the hemostasis valve.

18. The system of claim 15, wherein the engagement means comprises at least two teeth disposed upon the annular wall and extending radially inward for engaging the opposing mating component of the hemostasis valve.

19. The system of claim 18, wherein the annular wall is deformable to allow the at least two teeth to slide past the opposing mating component of the hemostasis valve.

20. The system of claim 15, wherein
the sheath is a splittable sheath;
the sliding connector has a distal range of motion such that the sliding connector can be moved to a position entirely disengaged from the hemostasis valve; and
the sliding connector is adapted to be split in half axially in response to radial force imparted by the nipple on an inner surface of the sliding connector defined by the connector lumen as the splittable sheath is split.
